(19) United States
(12) Patent Application Publication
(10) Pub. No.: US 2009/0292153 A1
(43) Pub. Date: Nov. 26, 2009

(54) Oxydative dehydrogenation of paraffins

(75) Inventors: Haiyong Cai, Calgary (CA); Andrzej Krzywicki, Calgary (CA)

Correspondence Address: Kenneth H. Johnson;Patent Attorney, P.O. Box 630708, Houston, TX, 77263, US

(73) Assignee: NOVA CHEMICALS (INTERNATIONAL) S.A

(21) Appl. No.: 12/454,075

(22) Filed: May 12, 2009

(30) Foreign Application Priority Data
May 20, 2008 (CA) 2,631,942

(51) Int. Cl. C07C 5/333

(52) U.S. Cl. 585/663; 585/661

(57) ABSTRACT

Lower paraffins may be oxidatively dehydrogenated in the presence of an oxidative dehydrogenation catalyst and one or more reducible metal oxides selected from the group consisting of NiO, Ce₂O₃, Fe₂O₃, TiO₂, Cr₂O₃, V₂O₅, WO₃, and mixtures thereof optionally with alumina may be dehydrogenated (regenerated) under milder conditions in a safe manner with the oxygen being provided by the metal oxides rather than direct addition of oxygen to the reactor.

### FIELD OF THE INVENTION

The present invention relates to the oxidative dehydrogenation of paraffins to olefins. More particularly the present invention relates to the catalytic oxidative dehydrogenation of paraffins to olefins in the presence of a catalyst and a regenerable metallic oxide or oxidant.

### BACKGROUND OF THE INVENTION

Currently paraffins, particularly aliphatic paraffins, are converted to olefins using thermal cracking technology. Typically the paraffins are passed through a furnace tube heated to at least 800° C., typically from about 850° C. to the upper working temperature of the alloy for the furnace tube, generally about 950° C. to 1000° C., for a period of time in the order of milliseconds to a few seconds. The paraffin molecule loses hydrogen and one or more unsaturated bonds are formed to produce an olefin. The current thermal cracking processes are not only cost intensive to build and operate but also energy intensive due to the substantial heat requirement for the endothermic cracking reactions. As a result, significant amounts of CO₂ are produced from the operation of these cracking furnaces.

Alternatively, it is known that olefins can be produced by reactions between paraffins with oxygen. However, this technology has not been commercially practiced for a number of reasons including the potential for an explosive mixture of oxygen and paraffin at an elevated temperature. For satisfactory conversion of paraffins to olefins, the required oxygen in the feed mixture should be typically higher than the maximum allowable level before entering the explosion range. Another reason is the requirement of either front end oxygen separation or a back end nitrogen separation, which often brings the overall process economy into negative territory. Therefore, solutions to address these issues are being sorted in various directions.

There are a number of United States patents assigned to Petro-Tex Chemical Corporation issued in the late 1960's that disclose the use of various ferrites in a steam cracker to produce olefins from paraffins. The patents include U.S. Pat. Nos. 3,420,911 and 3,420,912 in the names of Woskow et al. The patents teach introducing ferrites such as zinc, cadmium, and manganese ferrites (i.e. mixed oxides with iron oxide). The ferrites are introduced into a dehydrogenation zone at a temperature from about 250° C. up to about 750° C. at pressures less than 100 psi (689.476 kPa) for a time less than 2 seconds, typically from 0.005 to 0.9 seconds. The reaction appears to take place in the presence of steam that may tend to shift the equilibrium in the “wrong” direction. Additionally the reaction does not take place in the presence of a catalyst.

GB 1,213,181, which seems to correspond in part to the above Petro-Tex patents, discloses that nickel ferrite may be used in the oxidative dehydrogenation process. The reaction conditions are comparable to those of above noted Petro-Tex patents.

In the Petro-Tex patents the metal ferrite (e.g. M FeO₄ where, for example, M is Mg, Mn, Co, Ni, Zn or Cd) is circulated through the dehydrogenation zone and then to a regeneration zone where the ferrite is reoxidized and then fed back to the dehydrogenation zone.

Subsequent to the Petro-Tex patents a number of patents were published relating to the catalyst dehydrogenation of paraffins. However, these patents do not include the use of the ferrites of the Petro-Tex patents to provide a source of oxygen.

U.S. Pat. No. 6,891,075 issued May 10, 2005 to Liu, assigned to Symyx Technologies, Inc. teaches a catalyst for the oxidative dehydrogenation of a paraffin (alkane) such as ethane. The gaseous feedstock comprises at least the alkane and oxygen, but may also include diluents (such as argon, nitrogen, etc.) or other components (such as water or carbon dioxide). The dehydrogenation catalyst comprises at least about 2 weight % of NiO and a broad range of other elements preferably Nb, Ta, and Co. While NiO is present in the catalyst it does not appear to be the source of the oxygen for the oxidative dehydrogenation of the alkane (ethane).

U.S. Pat. No. 6,521,808 issued Feb. 18, 2003 to Ozkan, et al, assigned to the Ohio State University teaches sol gel supported catalysts for the oxidative dehydrogenation of ethane to ethylene. The catalyst appears to be a mixed metal system such as Ni—Co—Mo, V—Nb—Mo possibly doped with small amounts of Li, Na, K, Rb, and Cs on a mixed silica oxide/titanium oxide support. Again the catalyst does not provide the oxygen for the oxidative dehydrogenation; rather gaseous oxygen is included in the feed.

The present invention seeks to provide a simple process for the oxidative dehydrogenation of paraffins in the presence of a catalyst and a metal oxide or a mixture of metal oxides to provide oxygen for the process. The oxide may be regenerated and used again either by recycling through a regeneration zone or by using parallel beds so that the oxide may be regenerated by swinging the feed from an exhausted bed to a fresh bed and regenerating the oxide in the exhausted bed.

### SUMMARY OF THE INVENTION

The present invention provides a continuous process for the oxidative dehydrogenation of one or more C₂₋₁₀ alkanes comprising contacting said alkane with a bed of oxidative dehydrogenation catalyst on an inert support and a regenerable metallic oxidant composition at a temperature from 300° C. to 700° C., a pressure from 0.5 to 100 psi (3.447 to 689.47 kPa) and a residence time of the alkane in said bed of less than 5 seconds, wherein the oxidative dehydrogenation catalyst is selected from the group consisting of:

i) catalysts of the formula:

NiₓAₐB_{b}D_{d}Oₑ

wherein
x is a number from 0.1 to 0.9 preferably from 0.3 to 0.9, most preferably from 0.5 to 0.85, most preferably 0.6 to 0.8;
a is a number from 0.04 to 0.9;
b is a number from 0 to 0.5;
d is a number from 0 to 0.5;
e is a number to satisfy the valence state of the catalyst;
A is selected from the group consisting Ti, Ta, V, Nb, Hf, W, Y, Zn, Zr, Si, and Al or mixtures thereof;
B is selected from the group consisting of La, Ce, Pr, Nd, Sm, Sb, Sn, Bi, Pb, TL, IN, Te, Cr, Mn, Mo, Fe, Co, Cu, Ru, Rh, Pd, Pt, Ag, Cd, Os, Ir, Au, Hg and mixtures thereof; D is selected from the group consisting of Ca, K, Mg, Li, Na, Sr, Ba, Cs, and Rb and mixtures thereof; and
O is oxygen; and
   ii) catalysts of the formula

MO_{f}X_{g}Yₕ

wherein
X is selected from the group consisting of Ba, Ca, Cr, Mn, Nb, Ta, Ti, Te, V, W and mixtures thereof;
Y is selected from the group consisting of Bi, Ce, Co, Cu, Fe, K, Mg, V, Ni, P, Pb, Sb, Si, Sn, Ti, U and mixtures thereof;
f=1;
g is 0 to 2;
h=0 to 2, with the proviso that the total value of h for Co, Ni, Fe and mixtures thereof is less than 0.5; and
mixtures thereof to provide a weight ratio of oxidative dehydrogenation catalyst to metallic oxidant from 0.5:1 to 2:1 and said metallic oxidant is selected from the group consisting of NiO, Ce₂O₃, Fe₂O₃, TiO₂, Cr₂O₃, V₂O₅, WO₃ and mixtures thereof and mixtures of NiO, Ce₂O₃, Fe₂O₃, TiO₂, Cr₂O₃, V₂O₅, WO₃ and mixtures thereof and aluminum in a weight ratio from 0.5:1 to 1:1.5.

The above process is conducted in the absence of a gaseous oxygen feed.

### BRIEF DESCRIPTION OF THE DRAWINGS

FIG. 1 is a schematic drawing of a moving bed oxidative dehydrogenation process of the present invention.

### DETAILED DESCRIPTION

The oxidative dehydrogenation catalyst of the present invention may be selected from the group consisting of:

i) catalysts of the formula:

NiₓAₐB_{b}D_{d}Oₑ

wherein
x is a number from 0.1 to 0.9 preferably from 0.3 to 0.9, most preferably from 0.5 to 0.85, most preferably 0.6 to 0.8;
a is a number from 0.04 to 0.9;
b is a number from 0 to 0.5;
d is a number from 0 to 0.0.5;
e is a number to satisfy the valence state of the catalyst;
A is selected from the group consisting Ti, Ta, V, Nb, Hf, W, Y, Zn, Zr, Si and Al or mixtures thereof; B is selected from the group consisting of La, Ce, Pr, Nd, Sm, Sb, Sn, Bi, Pb, TI, In, Te, Cr, Mn, Mo, Fe, Co, Cu, Ru, Rh, Pd, Pt, Ag, Cd, Os, Ir, Au, Hg and mixtures thereof; D is selected from the group consisting of Ca, K, Mg, Li, Na, Sr, Ba, Cs, and Rb and mixtures thereof; and
O is oxygen; and
   ii) catalysts of the formula:

MO_{f}X_{g}Yₕ

wherein
X is selected from the group consisting of Ba, Ca, Cr, Mn, Nb, Ta, Ti, Te, V, W and mixtures thereof;
Y is selected from the group consisting of Bi, Ce, Co, Cu, Fe, K, Mg V, Ni, P, Pb, Sb, Si, Sn, Ti, U and mixtures thereof;
f=1;
g is 0 to 2;
h is 0 to 2, with the proviso that the total value of h for Co, Ni, Fe and mixtures thereof is less than 0.5;
and mixtures thereof.

In one embodiment the catalyst is the catalyst of formula i) wherein x is from 0.5 to 0.85, a is from 0.15 to 0.5, b is from O to 0.1 and d is from O to 0.1. In catalyst i) typically A is selected from the group consisting of Ti, Ta, V, Nb, Hf, W, Zr, Si, Al and mixtures thereof, B is selected from the group consisting of La, Ce, Nd, Sb, Sn, Bi, Pb, Cr, Mn, Mo, Fe, Co, Cu, Ru, Rh, Pd, Pt, Ag, Cd, Os, Ir and mixtures thereof and D is selected from the group consisting of Ca, K, Mg, Li, Na, Ba, Cs, Rb and mixtures thereof.

In an alternative embodiment the catalyst is catalyst ii). In some embodiments of this aspect of the invention typically X is selected from the group consisting of Ba, Ca, Cr, Mn, Nb, Ti, Te, V, W and mixtures thereof, Y is selected from the group consisting of Bi, Ce, Co, Cu, Fe, K, Mg V, Ni, P, Pb, Sb, Sn, Ti and mixtures thereof.

Typically the oxidative dehydrogenation catalyst is on a support such as alumina or silica. The catalyst loading on the support may range from 0.1 to 5 weight % of the support.

The metal oxide that provides the source of oxygen for the oxidative dehydrogenation may be NiO, Ce₂O₃, Fe₂O₃, TiO₂, Cr₂O₃, V₂O₅, WO₃ and mixtures thereof and the weight ratio of oxidative dehydrogenation catalyst to metallic oxidant is from 0.8:1 to 1:0.8. In a further embodiment of the invention the metal oxide is a mixture of NiO, Ce₂O₃, Fe₂O₃, TiO₂, Cr₂O₃, V₂O₅, WO₃ and alumina in and alumina in a weight ratio 0.8:1 to 1:0.8 and the oxidative dehydrogenation catalyst is used in an amount to provide a weight ratio of oxidative dehydrogenation catalyst to metallic oxidant from 0.8:1 to 1:0.8.

Typically the reaction is conducted at a temperature from 300° C. to 600° C. preferably from 400° C. to 600° C., pressure is from 15 to 50 psi (103.4 to 344.73 kPa) and the residence time of the paraffin (alkane) in said bed is less than 5 preferably less than 2 seconds, generally less than 1 second. The paraffin is typically selected from the group consisting of C₂₋₈, preferably C₂₋₄, straight chained paraffins (alkanes). Desirably the paraffin is selected from propane and ethane, preferably ethane. It is desirable to use a single paraffin having a high degree of purity, typically more than 95% pure, preferably more than 98% pure.

The process of the present invention may be continuous, or a batch or semi batch process.

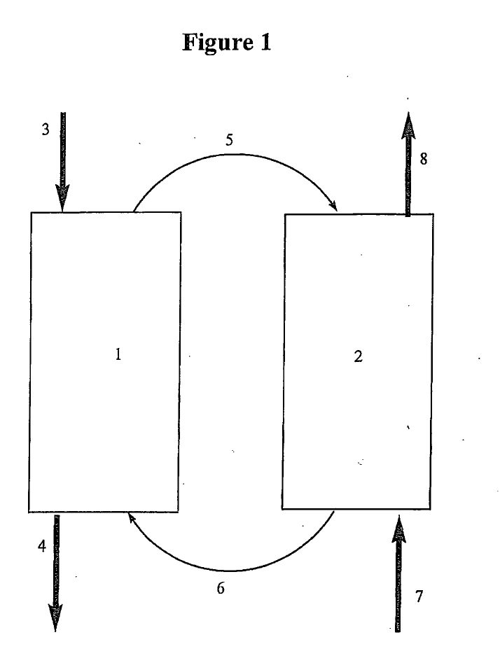

FIG. 1 is a schematic representation of one configuration of the reactors in which the present invention may be conducted. In FIG. 1 there are two vessels, 1 and 2, in parallel arrangement. In vessel 1 there is a bed, preferably of fluidized oxidative dehydrogenation catalyst and an oxide or a simple moving bed. A stream of reactants 3, typically paraffin, optionally with an inert gas such as nitrogen, such as ethane enters reactor 1. The paraffin undergoes oxidative dehydrogenation and the metal oxide or the oxide mixture gives up oxygen. A stream 4 of alkene such as ethylene leaves the reactor. The bed or at least the metal oxide component is moved from reactor 1 to reactor 2 by line 5. In reactor 2 an oxygen containing stream 7 such as air enters the reactor. The oxygen in the feed stream contacts the depleted oxide or the oxide mixture and regenerates it by oxidation. The regenerated oxide or the oxide mixture and optionally the oxidative dehydrogenation catalyst are then returned to reactor 1 by line 6.

In some embodiments both the oxidative dehydrogenation catalyst and the metal oxide are transferred between the reactors. However, it is also possible to use a segregated or partitioned bed, for example with a porous divider such as a fine screen or a membrane permeable to oxygen. In such an embodiment only the metal oxide is transferred between the reactors.

In an alternate embodiment there are two or more reactors in parallel arrangement. The reactor beds comprise a mixture of oxidative dehydrogenation catalyst and metal oxide or oxide mixture. When the metal oxide is nearing depletion the paraffin feed is switched to a different reactor. The exhausted reactor is vented and a feed of an oxygen containing stream passes through the bed to regenerate the metal oxide or oxide mixture. When the metal oxide or oxide mixture is regenerated the bed is ready to commence the reaction again.

The regeneration of the metal oxide generally takes place at low temperatures, typically from about 200° C. to 650° C., preferably from about 300° C. to 650° C., desirably from 400° C. to 550° C., at pressures less than 10132.5 kPa (100 atm), typically less than 5066.25 kPa (50 atm), generally from 1013.25 kPa (10 atm) to 101.32 kPa (1 atm). The feed stream is rich in oxygen and typically is air although pure oxygen could be used or a mixture of oxygen and nitrogen. The time to regenerate the oxide will depend on the mass of oxide and oxide mixture in the bed and the rate of regeneration of the oxide. This can be determined by one of ordinary skill in the art relatively easily by oxidizing depleting and regenerating a relatively small sample of oxide.

As noted above the present invention is practiced at lower temperatures than the current cracking process reducing energy costs and greenhouse gases. Additionally if the feed is a relatively pure paraffin (e.g. greater than 95% purity) and the oxidative dehydrogenation catalyst has a fairly high selectivity (e.g. greater than 95%, preferably greater than 98%), the separation costs at the back end of the oxidative dehydrogenation may also be reduced over a conventional cracking process in which several cryogenic separations may be required.

The present invention will be demonstrated by the following non-limiting examples.

### EXAMPLES

### Example 1

A selection of metal powders including Fe, Ni and Cr were oxidized by air in a thermal balance. The oxidation started at about 300° C. For iron complete oxidation was reached at 600° C. with Fe₂O₃ being the end product. However, the weight gains for Ni and Cr suggest incomplete oxidation in the same oxidation period. Further experimental tests were carried out to these oxides and the results show that both Fe₂O₃ and NiO can be reduced by ethane. However NiO appears to have a more favorable temperature range (400° C. to 600° C.). This example confirms that oxidation of metal (Ni) by air and reduction of the metal oxide (NiO) by ethane can take place in the same or similar temperature range for oxidative dehydrogenation. This confirms the required cycle between metal oxidation and the reduction of the metal oxide.

### Example 2

Powders of Ni of a particle size less than 250 mesh mixed with an equal amount of alumina of 140-200 mesh were packed in the reactor of a micro reaction unit (MRU). The reactor bed had a volume of 2 ml. The reactor bed was heated at about 10° C./min to 600° C. under 50 sccm (standard cubic centimeters) N₂ purge. At 600° C. a 25 sccm flow of air was admitted into the packed bed for 150 minutes in order to oxidize the Ni. Then the reactor was cooled in 50 sccm of N₂ to 450° C. and held at this temperature for 30 minutes to ensure complete removal of oxygen from the reactor. At the end of the cooling/purging period a stream of ethane was admitted to the reactor at a rate of 50 sccm and the composition in mole % of the reactor effluent was analyzed by a gas chromatograph. Two experiments were carried out under identical conditions and the product compositions are shown in Table 1.

TABLE 1
Product Composition in the Absence of Ni/NiOx

| Run Time | | | | | | |
|---|---|---|---|---|---|---|
| min | CH₄ | C₂H₆ | C₂H₄ | C₃H₆ | O₂ | CO₂ |
| 5 | 3.16 | 93.42 | 0.49 | 0.00 | 1.38 | 1.55 |
| 15 | 0.25 | 99.22 | 0.27 | 0.00 | 0.09 | 0.18 |
| 60 | 0.06 | 99.69 | 0.11 | 0.00 | 0.07 | 0.08 |
| 120 | 0.08 | 99.57 | 0.20 | 0.00 | 0.12 | 0.04 |
| 180 | 0.06 | 99.65 | 0.19 | 0.00 | 0.10 | 0.00 |
| 240 | 0.06 | 99.62 | 0.20 | 0.00 | 0.13 | 0.00 |
| 300 | 0.04 | 99.66 | 0.21 | 0.00 | 0.09 | 0.00 |
| 5 | 0.32 | 97.23 | 0.40 | 0.00 | 1.34 | 0.72 |
| 15 | 0.30 | 99.21 | 0.28 | 0.00 | 0.10 | 0.12 |
| 60 | 0.01 | 99.68 | 0.10 | 0.00 | 0.11 | 0.11 |
| 120 | 0.02 | 99.74 | 0.04 | 0.00 | 0.13 | 0.08 |
| 180 | 0.02 | 99.82 | 0.04 | 0.00 | 0.13 | 0.00 |
| 240 | 0.01 | 99.80 | 0.05 | 0.00 | 0.14 | 0.00 |
| 300 | 0.01 | 99.83 | 0.06 | 0.00 | 0.11 | 0.00 |

The results show a maximum less than 0.50 mole % of ethylene is formed under the reaction conditions.

### Example 3

Example 2 was repeated except that in addition to the Ni alumina powder the reactor contained an oxidative dehydrogenation catalyst (V—Mo—Nb—Te—Ox weight ratios) in a weight ratio of Ni:alumina:oxidative dehydrogenation catalyst of 2:2:1. Two repeat experiments were run using the same conditions as in Example 2. The effluent was analyzed for its composition using a gas chromatograph. The results are shown in Table 2. In Table 2 the amounts of the components are shown in mole %.

TABLE 2
Product Composition in the Presence of Ni/NiOx

| Run Time | | | | | | |
|---|---|---|---|---|---|---|
| Min | CH₄ | C₂H₆ | C₂H₄ | C₃H₆ | O₂ | CO₂ |
| 5 | 0.11 | 96.87 | 1.73 | 0.00 | 0.88 | 0.41 |
| 15 | 0.02 | 98.76 | 0.94 | 0.01 | 0.09 | 0.16 |
| 60 | 0.02 | 99.11 | 0.70 | 0.01 | 0.06 | 0.10 |
| 120 | 1.48 | 97.26 | 0.09 | 0.00 | 0.09 | 1.09 |
| 180 | 0.51 | 98.99 | 0.08 | 0.00 | 0.11 | 0.31 |
| 240 | 0.24 | 99.31 | 0.11 | 0.00 | 0.11 | 0.23 |
| 300 | 0.23 | 99.31 | 0.13 | 0.00 | 0.11 | 0.22 |
| 5 | 0.06 | 96.82 | 1.92 | 0.01 | 0.61 | 0.57 |
| 15 | 0.01 | 98.72 | 1.01 | 0.01 | 0.04 | 0.21 |
| 60 | 0.03 | 99.34 | 0.46 | 0.00 | 0.05 | 0.12 |
| 120 | 0.06 | 99.26 | 0.38 | 0.00 | 0.10 | 0.21 |
| 180 | 0.12 | 98.89 | 0.41 | 0.00 | 0.12 | 0.46 |
| 240 | 0.15 | 98.83 | 0.57 | 0.01 | 0.10 | 0.35 |
| 300 | 0.18 | 98.70 | 0.72 | 0.01 | 0.11 | 0.28 |

These results show an enhancement of ethylene yield when the oxidative dehydrogenation catalyst is present. The initial ethylene yields were close to 2 mole % compared to less than 0.50 mole % in the absence of the oxidative dehydrogenation catalyst. With increasing time the ethylene yield decreases indicating the oxygen present in the oxide is being depleted. These results, albeit low, do confirm that oxygen stored as metallic oxides was released and reacted with the ethane in the presence of the oxidative dehydrogenation catalyst without the addition of a gaseous stream containing oxygen to the reactor.

What is claimed is:

1. A process for the oxidative dehydrogenation of one or more C₂₋₁₀ alkanes comprising contacting said alkane with a bed of oxidative dehydrogenation catalyst on an inert support and a regenerable metallic oxidant composition at a temperature from is 300° C. to 700° C., a pressure from 0.5 to 100 psi (3.447 to 689.47 kPa) and a residence time of the alkane in said bed of less than 2 seconds, wherein the oxidative dehydrogenation catalyst is selected from the group consisting of:
i) catalysts of the formula:
NiₓAₐB_{b}D_{d}Oₑ
wherein
x is a number from 0.1 to 0.9, preferably from 0.3 to 0.9, most preferably from 0.5 to 0.85, most preferably 0.6 to 0.8;
a is a number from 0.04 to 0.9;
b is a number from 0 to 0.5;
d is a number from 0 to 0.0.5;
e is a number to satisfy the valence state of the catalyst;
A is selected from the group consisting Ti, Ta, V, Nb, Hf, W, Y, Zn, Zr, Si and Al or mixtures thereof;
B is selected from the group consisting of La, Ce, Pr, Nd, Sm, Sb, Sn, Bi, Pb, TI, In, Te, Cr, Mn, Mo, Fe, Co, Cu, Ru, Rh, Pd, Pt, Ag, Cd, Os, Ir, Au, Hg, and mixtures thereof;
D is selected from the group consisting of Ca, K, Mg, Li, Na, Sr, Ba, Cs, and Rb and mixtures thereof; and
O is oxygen; and
ii) catalysts of the formula
MO_{f}X_{g}Yₕ
wherein
X is selected from the group consisting of Ba, Ca, Cr, Mn, Nb, Ta, Ti, Te, V, W and mixtures thereof;
Y is selected from the group consisting of Bi, Ce, Co, Cu, Fe, K, Mg, V, Ni, P, Pb, Sb, Si, Sn, Ti, U, and mixtures thereof;
f=1;
g is 0 to 2;
h=0 to 2, with the proviso that the total value of h for Co, Ni, Fe and mixtures thereof is less than 0.5; and
mixtures thereof to provide a weight ratio of oxidative dehydrogenation catalyst to metallic oxidant from 0.5:1 to 2:1 and said metallic oxidant is selected from the group consisting of NiO, Ce2O3, Fe2O3, TiO2, Cr2O3, V2O5, WO3 and mixtures thereof and mixtures of such oxides and aluminum in a weight ratio from 0.5:1 to 1:1.5.

2. The process according to claim 1 wherein the temperature is from 400° C. to 600° C., the pressures is from 15 to 50 psi (103.4 to 344.73 kPa) and the residence time of the paraffin in said bed is less than 5 seconds.

3. The process according to claim 2 wherein the metallic oxidant is NiO, Ce2O3, Fe2O3, TiO2, Cr2O3, V2O5, WO3 and mixtures thereof and the weight ratio of oxidative dehydrogenation catalyst to metallic oxidant is from 0.8:1 to 1:0.8.

4. The process according to claim 2 wherein the metallic oxidant is a mixture of NiO, Ce2O3, Fe2O3, TiO2, Cr2O3, V2O5, WO3 and mixtures thereof and alumina in a weight ratio 0.8:1 to 1:0.8 and the oxidative dehydrogenation catalyst is used in an amount to provide a weight ratio of oxidative dehydrogenation catalyst (and support) to metallic oxidant from 0.8:1 to 1:0.8.

5. The process according to claim 3 wherein there are two or more separate fixed beds in parallel arrangement and the metallic oxidant in one or more beds is regenerated by passing an oxygen containing gas stream therethrough while maintaining at least one bed in operation.

6. The process according to claim 3 wherein the bed is a fluidized bed or a simple moving bed and a mixture of oxidative dehydrogenation catalyst and metallic oxide is removed from said bed and the metallic oxidant is regenerated by passing an oxygen containing gas stream therethrough and the mixture is returned to the fluidized bed.

7. The process according to claim 3 wherein the bed is a segregated bed with the metallic oxide separated from the oxidative dehydrogenation catalyst by an oxygen permeable membrane and at least a portion of the metallic oxide is removed from said bed and regenerated by passing an oxygen containing gas stream therethrough and the metallic oxide is returned to the bed.

8. The process according to claim 4 wherein there are two or more separate fixed beds in parallel arrangement and the metallic oxidant in one or more beds is regenerated by passing an oxygen containing gas stream therethrough while maintaining at least one bed in operation.

9. The process according to claim 4 wherein the bed is a fluidized bed or a simple moving bed and a mixture of oxidative dehydrogenation catalyst and metallic oxide is removed from said bed and the metallic oxidant is regenerated by passing an oxygen containing gas stream therethrough and the mixture is returned to the fluidized bed.

10. The process according to claim 4 wherein the bed is a segregated bed with the metallic oxide separated from the oxidative dehydrogenation catalyst by an oxygen permeable membrane and at least a portion of the metallic oxide is removed from said bed and regenerated by passing an oxygen containing gas stream therethrough and the metallic oxide is returned to the bed.

11. The process according to claim 5 wherein the alkane is ethane and the oxidative dehydration catalyst is catalyst i) wherein x is from 0.5 to 0.85, a is from 0.15 to 0.5, b is from 0 to 0.1 and d is from 0 to 0.1.

12. The process according to claim 6 wherein the alkane is ethane and the oxidative dehydration catalyst is catalyst i) wherein x is from 0.5 to 0.85, a is from 0.15 to 0.5, b is from 0 to 0.1 and d is from 0 to 0.1.

13. The process according to claim 7 wherein the alkane is ethane and the oxidative dehydration catalyst is catalyst i) wherein x is from 0.5 to 0.85, a is from 0.15 to 0.5, b is from 0 to 0.1 and d is from 0 to 0.1.

14. The process according to claim 8 wherein the alkane is ethane and the oxidative dehydration catalyst is catalyst i) wherein x is from 0.5 to 0.85, a is from 0.15 to 0.5, b is from 0 to 0.1 and d is from 0 to 0.1.

15. The process according to claim 9 wherein the alkane is ethane and the oxidative dehydration catalyst is catalyst i) wherein x is from 0.5 to 0.85, a is from 0.15 to 0.5, b is from 0 to 0.1 and d is from 0 to 0.1.

16. The process according to claim 10 wherein the alkane is ethane and the oxidative dehydration catalyst is catalyst i) wherein x is from 0.5 to 0.85, a is from 0.15 to 0.5, b is from 0 to 0.1 and d is from 0 to 0.1.

17. The process according to claim 5 wherein the alkane is ethane and the oxidative dehydration catalyst is catalyst ii).

18. The process according to claim 6 wherein the alkane is ethane and the oxidative dehydration catalyst is catalyst ii).

19 The process according to claim 7 wherein the alkane is ethane and the oxidative dehydration catalyst is catalyst ii).

20. The process according to claim 8 wherein the alkane is ethane and the oxidative dehydration catalyst is catalyst ii).

21. The process according to claim 9 wherein the alkane is ethane and the oxidative dehydration catalyst is catalyst ii).

22. The process according to claim 10 wherein the alkane is ethane and the oxidative dehydration catalyst is catalyst ii).
